SEQUENCE LISTING (2 sequences)

SEQ ID NO: 1 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 2 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

(19) United States
(12) Patent Application Publication
(10) Pub. No.: US 2024/0358721 A1
(43) Pub. Date: Oct. 31, 2024

(54) GASDERMIN-D INHIBITORS AND USES THEREOF FOR TREATING PULMONARY VASO-OCCLUSION

(71) Applicant: UNIVERSITY OF PITTSBURGH-OF THE COMMONWEALTH SYSTEM OF HIGHER EDUCATION, Pittsburgh, PA (US)

(72) Inventor: Prithu SUNDD, Pittsburgh, PA (US)

(21) Appl. No.: 18/688,496

(22) PCT Filed: Sep. 1, 2022
(86) PCT No.: PCT/US2022/075822

§ 371 Date: Mar. 1, 2024

Related U.S. Application Data
(60) Provisional application No. 63/239,598, filed on Sep. 1, 2021.

(51) Int. Cl. A61K 31/635; A61K 31/145; A61K 31/553; A61K 31/713; A61K 39/00; A61P 9/12; C07K 16/28; C12N 9/22; C12N 15/11

(52) U.S. Cl. CPC A61K 31/635; A61K 31/145; A61K 31/553; A61K 31/713; A61P 9/12; C07K 16/2854; C12N 9/22; C12N 15/111; A61K 2039/505; C12N 2310/122; C12N 2310/14; C12N 2310/20

(57) ABSTRACT

The present disclosure relates to methods for treating pulmonary vaso-occlusion in a subject comprising administering to the subject a gasdermin-D inhibitor. In some examples. the disclosed methods are effective at treating subjects resistant to P-selectin-based therapy.

### CROSS-REFERENCE TO RELATED APPLICATIONS

This application claims the benefit of U.S. Provisional Application No. 63/239,598, filed Sep. 1, 2021, which is expressly incorporated herein by reference in its entirety.

### STATEMENT REGARDING FEDERALLY SPONSORED RESEARCH

This invention was made with government support under grant numbers HL128297 and HL141080 awarded by the National Institutes of Health (NIH). The government has certain rights in the invention.

### FIELD

The present disclosure relates to the field of gasdermin-D inhibitors and uses thereof for treating pulmonary vaso-occlusion.

### BACKGROUND

Sickle Cell Disease (SCD) is an autosomal-recessive-genetic disorder that affects millions of people world-wide [Rees, D. C. et al., (2010); Mortality, G.B.D. & Causes of Death, C. (2015); Kato, G. J., et al. (2018)]. Sickle Cell Anemia, the most common form of SCD is caused by a homozygous mutation (SS) in the β-globin gene [Rees, D. C. et al., (2010); Kato, G. J., et al. (2018)]. The mutant hemoglobin (HbS) polymerizes under hypoxic conditions, leading to erythrocyte sickling, vaso-occlusion and hemolysis [Sundd, P., Gladwin, M. T. & Novelli, E. M. (2019); Telen, M. J., Malik, P. & Vercellotti, G. M. (2019); Zhang, D., et al. (2015); Hidalgo, A., et al. (2009)]. Vaso-occlusion contributes to the development of acute systemic painful vaso-occlusive episode, which is the primary reason for hospitalization of SCD patients [Rees, D. C., et al., 2010]. What is needed are compositions and methods for treating and preventing SCD and vaso-occlusion. The compositions and methods disclosed herein address these and other needs.

### SUMMARY

Disclosed herein is a method for treating pulmonary vaso-occlusion in a subject in need comprising administering a therapeutically effective amount of a gasdermin-D inhibitor to the subject. This method has been shown to be surprisingly effective at treating, preventing, and/or ameliorating pulmonary vaso-occlusion in a subject in need. In some embodiments, the subject in need is a patent with sickle cell disease. In some embodiments, the gasdermin-D inhibitor is selected from the group consisting of LDC7559, necrosulfonamide, and disulfiram. In some embodiments, the gasdermin-D inhibitor is a gasdermin-D gene editing tool that decreases the levels of a gasdermin-D polypeptide in the subject. In some embodiments, the method of any preceding aspect further comprises administering to the subject a therapeutically effective amount of a P-selectin inhibitor. The P-selectin inhibitor can be administered concurrently or sequentially with the gasdermin-D inhibitor.

P-selectin antibody therapy can reduce hospitalization of patients with SCD by ˜50%, indicating that an unknown P-selectin-independent mechanism promotes remaining vaso-occlusive events. The method disclosed herein is shown to be surprisingly effective at treating, preventing, and/or ameliorating pulmonary vaso-occlusion in a subject in need, wherein the subject is resistant to a P-selectin inhibitor.

Accordingly, in some aspects, disclosed herein is a method of treating pulmonary vaso-occlusion in a subject in need, comprising administering a therapeutically effective amount of a gasdermin-D inhibitor to the subject, wherein the subject is resistant to a P-selectin inhibitor. In some embodiments, the subject has sickle cell disease.

### DESCRIPTION OF DRAWINGS

FIGS. 1A-1P show that inhibiting GSDMD-pathway ameliorates P-selectin-independent lung vaso-occlusion in SCD. FIG. 1A shows experimental scheme: SCD or SCD-Selp^{-/-} mice and WT or Gsdmd^{-/-} mice were IV administered 10 μmole/kg oxy-Hb (10 oxy-Hb) and 20 μmole/kg oxy-Hb (20 oxy-Hb), respectively without or with 10 mg/kg GSDMD inhibitor (LDC7559) or 0.004 μmol/kg pan-caspase inhibitor (Z-VAD-FMK) or 20 mg/kg GSDMD inhibitor Necrosulfonamide (NSA). Quantitative fluorescence intravital lung microscopy (qFILM) was used to assess the absence or presence of platelet-neutrophil aggregate mediated pulmonary vaso-occlusion. Pulmonary microcirculation (pseudo-colored purple), neutrophils (red) and platelets (pseudo-colored green) were labeled in vivo by IV administration of FITC dextran, AF546-anti-Ly6G Ab and V450-anti-CD49b Ab, respectively. Representative qFILM images are shown in FIGS. 1B-1E. FIG. 1B shows that representative qFILM image reveals five neutrophil-platelet aggregates (marked with dotted white ellipses) occluding pulmonary arteriole-bottleneck in the lung of an SCD mouse IV administered 10 μmole/kg oxy-Hb (10 oxy-Hb). Representative qFILM images and corresponding Movies reveal absence of lung vaso-occlusion and significantly improved blood flow (evident by rapidly transiting erythrocytes (dark cells)) in the lung of an SCD mouse IV administered 10 oxy-Hb+Z-VAD-FMK (FIG. 1C), 10 oxy-Hb+LDC7559 (FIG. 1D) and 10 oxy-Hb+NSA (FIG. 1E). White arrows denote the direction of blood flow within the pulmonary arterioles. Alveoli are marked with asterisks. Scale bars 20 μm. The diameter of pulmonary arteriole in B-E is ˜22 μm, 32 μm, 31.5 μm, and 31 μm, respectively. FIGS. 1F-1K show that pulmonary vaso-occlusions (PVOs) were compared between treatment groups using following two parameters: Number of PVOs per Field of View (#PVOs/FOV) and number of large PVOs (with area>1000 μm²) per FOV. Refer Online Data Supplement for details. Both #PVOs/FOV and #PVOs (with area>1000 μm²) per FOV were significantly less in SCD mice IV administered (FIGS. 1F and 1G) 10 oxy-Hb+Z-VAD-FMK (n=4 mice; 51 FOVs) or (FIGS. 1H and 1I) 10 oxy-Hb+LDC7559 (n=4 mice; 41 FOVs) or (FIG. 1J and 1K) 10 oxy-Hb+NSA (n=3 mice; 38 FOVs) than SCD mice IV administered 10 oxy-Hb alone (n=5 mice; 75 FOVs). Both (FIG. 1L) #PVOs/FOV and (FIG. 1M) #PVOs (with area>1000 μm²) per FOV were significantly less in Gsdmd^{-/-} (n=3 mice; 28 FOVs) than littermate WT mice (n=3 mice; 31 FOVs) IV administered 20 oxy-Hb. (FIG. 1N) #PVOs/FOV were significantly reduced and (O) #PVOs (with area>1000 μm²) per FOV were absent in SCD-Selp^{-/-} mice IV administered 10 oxy-Hb 1 LDC7559 (n=3 mice; 35 FOVs) compared with SCD-Selp^{-/-} mice IV administered 10 oxy-Hb alone (n=5 mice; 69 FOVs). A similar effect of disulfiram on PVOs in SCD-Selp^{-/-} mice shown in FIG. 4. The data for mice with SCD IV administered 10 oxy-Hb (n=5 mice; 75 FOVs) are included as black bars in FIGS. 1N and 1O for relative comparison. qFILM FOV size ˜65 536 μm². Data represent mean ±SE and are compared using Student t test. *P<0.05 (FIG. 1P) Schematic showing the main findings of the study. The sterile inflammatory milieu (DAMPs, IFN-I signaling, and ROS) in SCD promotes caspase-4 (humans) or caspase-11 (mouse)-dependent cleavage of neutrophil GSDMD into the active form GSDMD-NT, leading to the shedding of NETs in the liver microcirculation. Once shed, these NETs are carried by the blood as cNETs to the lung, where they promote neutrophil-platelet aggregation in the pulmonary arterioles, leading to PVO and lung injury (acute chest syndrome).

FIG. 2 shows that intravenous administration of 20 μmol/kg oxy-Hb triggered lung vaso-occlusion in wild-type but not Gsdmd^{-/-} mice. Experimental scheme is shown in FIG. 1A. Litter-mate wild type (WT) or Gsdmd^{-/-} mice were intravenously (IV) administered 20 μmole/kg oxy-Hb (20 oxy-Hb). Quantitative fluorescence intravital lung microscopy (qFILM) was used to assess the absence or presence of pulmonary vaso-occlusion. Pulmonary microcirculation (pseudo-colored purple) and neutrophils (red). Representative qFILM images show a large neutrophil aggregate (marked with dotted ellipse) occluding the arteriolar bottle-neck in the lung of a WT mouse (left panel) given IV 20 oxy-Hb, while such aggregates were absent in the lung microcirculation of a Gsdmd^{-/-} mouse (right panel) given IV 20 oxy-Hb. White arrows denote the direction of blood flow within the pulmonary arterioles. Alveoli are marked with asterisks. Scale bars 20 μm. The diameter of pulmonary arteriole in left and right panel is 28.6 and 31.8 μm, respectively. qFILM FOV size˜65,536 μm².

FIG. 3 shows that P-selectin deficiency attenuates lung vaso-occlusion by ˜50% in SCD mice. SCD or SCD-Selp^{-/-} mice were intravenously (IV) administered 10 μmole/kg oxy-Hb (10 oxy-Hb). Quantitative fluorescence intravital lung microscopy (qFILM) was conducted to assess the absence or presence of pulmonary vaso-occlusion. Time-series of qFILM images were analyzed to quantify and compare pulmonary vaso-occlusions (PVOs) between treatment groups using following two parameters: Number of PVOs per Field of View (#PVOs/FOV) and number of large PVOs (with area>1000 μm²) per FOV. Refer Online Methods for details. Both #PVOs/FOV and #PVOs (with area>1000 μm²) per FOV were (significantly) reduced by ˜50% in SCD-Selp^{-/-} (n=5 mice; 69 FOVs) than SCD mice (n=5 mice; 75 FOVs) IV administered 10 oxy-Hb. qFILM FOV size˜65,536 μm². Data represents Mean±SE and compared using Students t-test. * denotes p<0.05.

FIGS. 4A-4B show that inhibiting GSDMD-pathway ameliorates P-selectin-independent lung vaso-occlusion in SCD. SCD-Selp^{-/-} mice were IV administered 10 μmole/kg oxy-Hb (10 oxy-Hb) without or with 10 mg/kg GSDMD inhibitor (Disulfiram). Quantitative fluorescence intravital lung microscopy (qFILM) was used to assess the absence or presence of pulmonary vasoocclusion using the same experimental scheme described in FIG. 1A. Pulmonary vasoocclusions (PVOs) were compared between treatment groups using following two parameters: Number of PVOs per Field of View (#PVOs/FOV) and number of large PVOs (with area>1000 μm²) per FOV. (FIG. 4A) #PVOs/FOV were significantly reduced and (FIG. 4B) #PVOs (with area>1000 μm²) per FOV were absent in SCD-Selp^{-/-} mice IV administered 10 oxy-Hb+Disulfiram (white bar; n=3 mice; 43 FOVs) compared to SCD-Selp^{-/-} mice IV administered 10 oxy-Hb alone (grey bar; n=5 mice; 69 FOVs). The data for SCD mice IV administered 10 oxy-Hb (n=5 mice; 75 FOVs) is included as black bars in FIGS. 4A and 4B for relative comparison. FOV size˜65,536 μm². Data represents Mean±SE and compared using one-way ANOVA with Games-Howell's multiple comparisons test. * denotes p<0.05.

FIGS. 5A-5J show that GSDMD promotes NETs generation and lung vaso-occlusion in SCD in a P-selectin-independent manner. (FIG. 5A) Experimental scheme: SCD or SCD-Selp^{-/-} mice were IV administered 10 μmole/kg oxy-Hb (10 oxy-Hb) without or with 10 mg/kg GSDMD inhibitors LDC75591 or Disulfiram. In FIGS. 5C-5D, venous blood was processed to generate PPP and cNETs detected in PPP using Imaging Flow Cytometry. Imaging Flow Cytometry data was quantified to estimate concentration of cNETs (#cNETs/μl of plasma). In FIGS. 5B, and 5E-5F, quantitative fluorescence intravital lung microscopy (qFILM) was used to detect arrival of circulating NETs (cNETs) within the lung microcirculation. Number of cNETs entering per FOV over a 1-minute duration (#cNETs/FOV/min) were quantified. In FIGS. 5G-5J, qFILM was used to assess the absence or presence of pulmonary vaso-occlusion using the experimental strategy described in FIG. 1A. Pulmonary vaso-occlusions (PVOs) were compared between treatment groups using following two parameters: Number of PVOs per Field of View (#PVOs/FOV) and number of large PVOs (with area>1000 μm²) per FOV. (FIG. 5B) #cNETs/FOV/min in SCD mice IV administered 10 oxy-Hb were not different from SCD-Selp^{-/-} mice IV administered 10 oxy-Hb. SCD IV Oxy-Hb (n=4 mice; 44 FOVs). SCD-Selp^{-/-} IV oxy-Hb (n=5 mice; 71 FOVs). (FIG. 5C) Plasma concentration of cNETs was not different between SCD and SCD-Selp^{-/-} mice (n=4 mice per group) IV administered 10 oxy-Hb. (FIG. 5D) Plasma concentration of cNETs was significantly less in SCD-Selp^{-/-} mice IV administered 10 oxy-Hb+LDC7559 (n=4 mice) than 10 oxy-Hb alone (n=4 mice). (FIG. 5E) #cNETs/FOV/min in the lung were significantly less (4-folds) in SCD-Selp^{-/-} mice IV administered 10 oxy-Hb+LDC7559 (n=3 mice; 35 FOVs) than 10 oxy-Hb alone (n=5 mice; 71 FOVs). (FIG. 5F) #cNETs/FOV/min in the lung were significantly less (4-folds) in SCD-Selp^{-/-} mice IV administered 10 oxy-Hb+Disulfiram (n=3 mice; 43 FOVs) than 10 oxy-Hb alone (n=5 mice; 71 FOVs). (FIG. 5G) #PVOs/FOV were significantly reduced and (FIG. 5H) #PVOs (with area>1000 μm²) per FOV were absent in SCD-Selp^{-/-} mice IV administered 10 oxy-Hb+LDC7559 (white bars; n=3 mice; 35 FOVs) compared to SCD-Selp^{-/-} mice IV administered 10 oxy-Hb alone (grey bars; n=5 mice; 69 FOVs). (FIG. 5I) #PVOs/FOV were significantly reduced and (FIG. 5J) #PVOs (with area>1000 μm²) per FOV were absent in SCD-Selp^{-/-} mice IV administered 10 oxy-Hb+Disulfiram (white bar; n=3 mice; 43 FOVs) compared to SCD-Selp^{-/-} mice IV administered 10 oxy-Hb alone (grey bar; n=5 mice; 69 FOVs). The data for SCD mice IV administered 10 oxy-Hb (n=5 mice; 75 FOVs) is included as black bars in FIGS. 5G-5J for relative comparison. FOV size˜65,536 μm². Data represents Mean±SE and compared using Students t-test (FIGS. 5B-5F) or one-way ANOVA with Games-Howell's multiple comparisons test (FIGS. 5G-5J). * denotes p<0.05.

### DETAILED DESCRIPTION

Cell-free oxy-Hb released during intravascular hemolysis is scavenged by the plasma haptoglobin, which chaperones it to the liver, spleen and bone marrow for clearance [Rees, D. C. et al., (2010); Kato, G. J., et al. (2018); Kato, G. J., Steinberg et al., (2017)]. However, SCD is associated with chronic depletion of haptoglobin, leading to impaired scavenging of cell-free oxy-Hb, which contributes to the development of sterile inflammation in SCD [Sundd, P. et al., (2019); Gladwin, M. T. et al., (2014); Sundd, P. et al., 2021)]. Based on this, SCD mice were systemically (IV) challenged with oxy-Hb to trigger vaso-occlusive crisis [Belcher, J. D., et al. (2014); Vats, R., et al. (2020)].

Intravital microscopy revealed that IV oxy-Hb promoted accumulation of NETs in the pulmonary arterioles and NETs-dependent lung vaso-occlusion by neutrophil-platelet aggregates, leading to development of lung injury in mice with SCD. It was surprising that these NETs were found to enter the lung via the pulmonary arterioles, suggesting that they originate in a non-pulmonary vascular bed. It was determined that these NETs were primarily shed by neutrophils in the liver microcirculation of mice with SCD and then transported by blood (as cNETs) to the lung, where they promoted lung vaso-occlusion. Indeed, cNETs were abundant in the peripheral blood of mice with SCD, and the levels were further elevated following challenge with IV oxy-Hb.

Importantly, shedding of cNETs and their embolization to lung were not affected by the absence of P-selectin, but significantly prevented following GSDMD inhibition in SCD-Selp2/2 mice given IV oxy-Hb. Finally, GSDMD inhibition completely abolished the remaining lung vaso-occlusion present in SCD-Selp2/2 mice given IV oxy-Hb. Taken together, the findings presented herein suggest for the first time that the sterile inflammatory milieu (DAMPs) in SCD promotes caspase-4/11-dependent activation of neutrophil-GSDMD, which leads to P-selectin-independent NETs generation in the liver. These NETs detach from parent neutrophils in the liver and then arrive as cNETs in the lung to promote occlusion of pulmonary arterioles by neutrophil-platelet aggregates. Most importantly, GSDMD inhibition also abrogates P-selectin-independent lung vaso-occlusion in SCD.

The findings shown herein introduce a novel paradigm that liver to lung translocation of DAMPs promotes lung injury in SCD and also identify a new GSDMD-mediated, P-selectin-independent mechanism of lung vaso-occlusion in SCD. In spite of recent advances in new therapies for SCD [Ataga, K. I., et al. (2017); Vichinsky, E., et al. (2019)], acute chest syndrome continues to be a major cause of morbidity among hospitalized SCD patients [Kato, G. J., et al. (2018)], but a pharmacological therapy to prevent its onset still remains clinically unavailable [Telen, M. J., et al., (2019)]. The current study is the first to highlight the therapeutic strategy of a multi-target therapy of blocking both P-selectin and GSDMD-dependent events, to prevent development of acute chest syndrome in high-risk SCD patients hospitalized with painful vaso-occlusive episodes. This study shows that such a combined anti-inflammatory approach can significantly control vasoocclusive painful episodes and secondary acute chest syndrome in patients with SCD.

Accordingly, disclosed herein methods and compositions for treating pulmonary vaso-occlusion in a subject in need thereof. The methods include administering to the subject a therapeutically effective amount of a gasdermin-D inhibitor. In some embodiments, the subject has a sickle cell disease. This method has been shown to be surprisingly effective at treating pulmonary vaso-occlusion.

### Terminology

As used herein, the terms “can,” “optionally,” and “can optionally” are used interchangeably and are meant to include cases in which the condition occurs as well as cases in which the condition does not occur. Thus, for example, the statement that a formulation “can include an excipient” is meant to include cases in which the formulation includes an excipient as well as cases in which the formulation does not include an excipient.

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, and the like. Administration includes self-administration and the administration by another.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., pulmonary vaso-occulsion or sickle cell disease). The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the terms “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Effective amount” encompasses, without limitation, an amount that can ameliorate, reverse, mitigate, or prevent a symptom or sign of a medical condition or disorder (e.g., pulmonary vaso-occlusion or acute chest syndrome). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

In the present invention, “specific for” and “specificity” mean selective binding. Accordingly, an antibody that is specific for one antigen selectively binds that antigen and not other antigens or not other antigens lacking epitope look-alikes.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of pulmonary vaso-occlusion and/or acute chest syndrome), during early onset (e.g., upon initial signs and symptoms of pulmonary vaso-occlusion and/or acute chest syndrome), after an established development of pulmonary vaso-occlusion and/or acute chest syndrome. Prophylactic administration can occur for several minutes to months prior to the manifestation of pulmonary vaso-occlusion or sickle cell disease.

In some instances, the terms “treat,” “treating,” “treatment,” and grammatical variations thereof, refers to mitigating pulmonary vaso-occlusion or acute chest syndrome, and/or related symptoms in a subject as compared with prior to treatment of the subject or as compared with incidence of such symptom in a general or study population. As used herein, “acute chest syndrome” refers to presentation with a new radiodensity on chest radiograph with respiratory symptoms. The acute chest syndrome can be mild, moderate or severe. In some embodiments, treatment of pulmonary vaso-occlusion results in an increase in pulmonary microcirculation or a reduction in pulmonary microcirculation obstruction. In some embodiments, treatment of pulmonary vaso-occlusion results in a decrease in chest pain associated with pulmonary microcirculation obstruction.

### Methods

Disclosed herein is a method for treating pulmonary vaso-occlusion in a subject in need comprising administering a therapeutically effective amount of a gasdermin-D inhibitor to the subject. This method has been shown to be surprisingly effective at treating, preventing, and/or ameliorating pulmonary vaso-occlusion in the subject.

The term “pulmonary vaso-occlusion” herein refers to the partial or complete blockage (occlusion) of the blood vessels that carry blood from the lung to the heart. In some embodiments, the pulmonary vaso-occlusion is an obstruction of the microcirculation.

The term “sickle cell disease” is a group of disorders that affects hemoglobin, the molecule in red blood cells that delivers oxygen to cells throughout the body. People with this disease have atypical hemoglobin molecules called hemoglobin S, which can distort red blood cells into a sickle, or crescent, shape. Pathophysiological studies have shown that the dense, dehydrated red blood cells can cause acute and chronic clinical manifestations of sickle cell disease, in which intravascular sickling in capillaries, small vessels, and large vessels leads to vaso-occlusion (e.g., pulmonary vaso-occlusion) and impaired blood flow with ischemic cell damage in a variety of organs and tissues. Accordingly, disclosed herein is a method of treating pulmonary vaso-occlusion in a subject in need comprising administering a therapeutically effective amount of a gasdermin-D inhibitor to the subject, wherein the subject has a sickle cell disease.

“Gasdermin-D” refers herein to a polypeptide that synthesizes and hydrolyzes cyclic adenosine 5′-diphosphate-ribose, and in humans, is encoded by the GSDMD gene. In some embodiments, the gasdermin-D polypeptide is that identified in one or more publicly available databases as follows: HGNC: 25697, NCBI Entrez Gene: 79792, Ensembl: ENSG00000104518, OMIM®: 617042, UniProtKB/Swiss-Prot: P57764. In some embodiments, the gasdermin-D polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The gasdermin-D polypeptide of SEQ ID NO: 1 may represent an immature or pre-processed form of mature gasdermin-D, and accordingly, included herein are mature or processed portions of the gasdermin-D polypeptide in SEQ ID NO: 1.

“Inhibitors” of expression or of activity are used to refer to inhibitory agents identified using in vitro assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 100%, 90%, 80%, 70%, 60% or 50%. The term “gasdermin-D inhibitor,” as used herein, refers to any molecule, compound, or substance that prevents, blocks, or impairs the function or activity of gasdermin-D. In some embodiments, the gasdermin-D inhibitor reduces the activity and/or expression of a gasdermin-D polypeptide by at least about 20%, at least about 50%, at least about 75%, at least about 80%, at least about 90%, at least about 95%, or at least about 99% in comparison to a control (untreated with inhibitors). It should be also understood that the gasdermin-D inhibitor can be an inhibitor of one or more other factors (e.g., one or more genes, proteins, mRNA) involved in the gasdermin-D pathway.

In some embodiments, the gasdermin-D inhibitor is selected from the group consisting of LDC7559, necrosulfonamide, and disulfiram. In some embodiments, the gasdermin-D inhibitor is LDC7559 as represented by the below Formula I or a pharmaceutically acceptable salt thereof. In some embodiments, the gasdermin-D inhibitor is necrosulfonamide as represented by the below Formula II or a pharmaceutically acceptable salt thereof. In some embodiments, the gasdermin-D inhibitor is disulfiram as represented by the below Formula III or a pharmaceutically acceptable salt thereof.

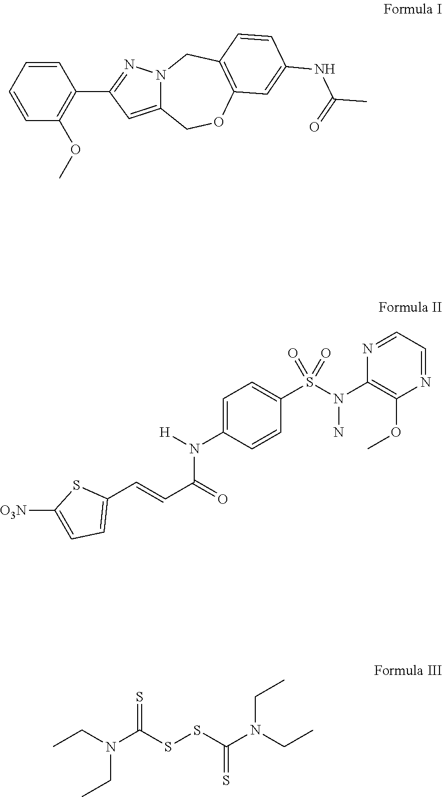

In some embodiments, the gasdermin-D inhibitor is a gasdermin-D gene editing tool. In some embodiments, the gasdermin-D gene editing tool is a small interfering RNA (siRNA), a short hairpin RNA (shRNA), CRISPR-Cas9, or CRISPR-Cas 13. The term “gene editing tool” refers to an agent or assay for inserting, deleting, modifying, or replacing one or more nucleotides in a nucleic acid (e.g., DNA or RNA) of a living organism. For example, the gene editing tool can reduce, prevent or block expression of the gene encoding gasdermin-D. Alternatively, the gene editing tool can reduce, prevent or block production of a gasdermin-D polypeptide following gene expression or decrease a level of a gasdermin-D polypeptide. Any suitable gene editing tool for gasdermin-D can be used in the methods described herein, including, but are not limited to, non-coding RNA (i.e., RNA that does not encode protein), such as small interfering RNAs (siRNAs), microRNAs (miRNAs), and short hairpin RNAs (shRNAs). siRNAs and miRNAs are central to RNA interference (RNAi), which is a biological process in which RNA molecules inhibit or reduce gene expression or translation by neutralizing targeted mRNA molecules. siRNA, also known as short interfering RNA or silencing RNA, is a class of double-stranded RNA molecules 20-25 base pairs in length, which interferes with the expression of specific genes with complementary nucleotide sequences by degrading mRNA after transcription, preventing translation (Agrawal et al., Microbiol. lvfol. Biol. Rev., 67(4): 657-668 (2003)). shRNAs are artificial RNA molecules with a tight hairpin tum that can be used to silence target gene expression via RNAi. CRISPR-Cas9 and CRISPR-Cas13 are gene editing tools using a CRISPR system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

The method disclosed herein can further comprise administering to the subject a therapeutically effective amount of a P-selectin inhibitor. In some embodiments, the P-selectin inhibitor is an antibody that specifically binds to P-selectin and wholly or partially inhibits P-selectin's activity.

“P-selectin” refers herein to a polypeptide that synthesizes and hydrolyzes cyclic adenosine 5′-diphosphate-ribose, and in humans, is encoded by the SELP gene. In some embodiments, the P-selectin polypeptide is that identified in one or more publicly available databases as follows: HGNC: 10721, NCBI Entrez Gene: 6403, Ensembl: ENSG00000174175, OMIM®: 173610, UniProtKB/Swiss-Prot: P16109. In some embodiments, the P-selectin polypeptide comprises the sequence of SEQ ID NO: 2, or a polypeptide sequence having at or greater than about 80%, about 85%, about 90%, about 95%, or about 98% homology with SEQ ID NO: 2, or a polypeptide comprising a portion of SEQ ID NO: 2. The P-selectin polypeptide of SEQ ID NO: 2 may represent an immature or pre-processed form of mature P-selectin, and accordingly, included herein are mature or processed portions of the P-selectin polypeptide in SEQ ID NO: 2.

The term “P-selectin inhibitor,” as used herein, refers to any molecule, compound, or substance that prevents, blocks, or impairs the function or activity of P-selectin. In some embodiments, the P-selectin inhibitor reduces the activity and/or expression of a P-selectin polypeptide by at least about 20%, at least about 50%, at least about 75%, at least about 80%, at least about 90%, at least about 95%, or at least about 99%. It should be also understood that the P-selectin inhibitor can be an inhibitor for one or more other factors (e.g., one or more genes, proteins, mRNA) involved in a P-selectin-related pathway. In some embodiments, the P-selectin inhibitor is crizanlizumab. In some embodiments, the crizanlizumab is ADAKVEO® produced by Novartis. ADAKVEO® was previously known as SEG101. Examples of P-selectin inhibitors included herein are those described in, for example, U.S. Pat. No. 9,068,001, which is incorporated by reference herein in its entirety.

The P-selectin inhibitor can be administered concurrently or sequentially with the gasdermin-D inhibitor. In some embodiments, the P-selectin inhibitor is administered after the gasdermin-D inhibitor. In some embodiments, the P-selectin inhibitor is administered prior to the gasdermin-D inhibitor. It should be understood and herein contemplated that the pulmonary vaso-occlusion in sickle cell disease can be caused by a P-selectin-dependent mechanism or a P-selectin-independent mechanism. Subjects having the pulmonary vaso-occlusion caused by a P-selectin-independent mechanism can be resistant to a P-selectin inhibitor therapy. The term “resistant” or the like, as used herein, refers to a reduced sensitivity to certain medicine (e.g., a P-selectin inhibitor) as compared to a positive control (e.g., a subject responding to the medicine (e.g., a P-selectin inhibitor)). In some embodiments, “resistant” refers to a reduction of about 100%, about 90%, about 80%, about 70%, about 60% or about 50%. Accordingly, disclosed herein is a method of treating pulmonary vaso-occlusion in a subject in need, said method comprises administering a therapeutically effective amount of a gasdermin-D inhibitor to the subject, and wherein the subject is resistant to a P-selectin inhibitor.

In some embodiments, the subject in need suffers from a condition or a combination of conditions selected from the group consisting of inflammatory pulmonary edema, inflammatory pulmonary infiltrates, impaired oxygenation, and hypoxemia. Accordingly, treatment using the methods disclosed herein results in at least one of improved survival, improved lung function, or reduced organ damage, reduced pulmonary vascular leakage, or a combination of any thereof. In some embodiments, treatment using the methods disclosed herein reduces at least one of inflammation, vasoconstriction, or platelet aggregation, or a combination of any thereof. In some embodiments, treatment using the methods disclosed herein reduces and/or prevents at least one of impaired blood flow (e.g., ischemia), blood coagulation, vascular inflammation, neutrophil number, thrombosis, ischemic cell damage, or organ damage, or a combination of any thereof. In some embodiments, treatment using the methods disclosed herein reduces and/or prevents pain or severity of the pain.

In certain embodiments, the methods of treating or preventing pulmonary vaso-occlusion comprises (i) administering a gasdermin-D inhibitor and (ii) evaluating whether a parameter or symptom has been ameliorated, wherein the parameter is selected from the group consisting of inflammation, vasoconstriction, platelet aggregation, lung function, organ (e.g., lung or kidney) damage, pulmonary vascular leakage, blood flow, blood coagulation, vascular inflammation, thrombosis, ischemic cell damage, presence of pain, severity of pain, frequency of occurrence of pulmonary vaso-occlusion, and duration of pulmonary vaso-occlusion.

In certain embodiments, the methods of treating or preventing pulmonary vaso-occlusion comprises (i) administering a P-selectin inhibitor, (ii) evaluating whether a parameter or symptom has been ameliorated, wherein the parameter is selected from the group consisting of inflammation, vasoconstriction, platelet aggregation, lung function, organ (e.g., lung or kidney) damage, pulmonary vascular leakage, blood flow, blood coagulation, vascular inflammation, thrombosis, ischemic cell damage, presence of pain, severity of pain, frequency of occurrence of VOC, and duration of VOC episodes, and (iii) administering a gasdermin-D inhibitor if the one or more of the parameters are not ameliorated.

As the timing of pulmonary vaso-occlusion can often not be predicted, it should be understood the disclosed methods of treating, preventing, reducing, and/or inhibiting a pulmonary vaso-occlusion, can be used prior to or following the onset of pulmonary vaso-occlusion to treat, prevent, inhibit, and/or mitigate any stage of the disease. The disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of pulmonary vaso-occlusion or a symptom thereof; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of pulmonary vaso-occlusion or a symptom thereof.

In some embodiments, the subject has sickle cell disease. It should be understood the disclosed methods of treating, preventing, reducing, and/or inhibiting a pulmonary vaso-occlusion, can be used prior to or following the onset of pulmonary vaso-occulsion, even prior to or during the onset of sickle cell crisis, or a blockage of blood flow causing pain.

Dosing frequency for the vector or the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, or daily. Administration can also be continuous and adjusted to maintaining a level of the compound within any desired and specified range.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Liver to Lung Transport of NETs Promotes Gasdermin-D-Dependent Lung Injury in Sickle Cell Disease

Clinical evidence suggests that acute chest syndrome, a type of acute lung injury and one of the leading causes of mortality among SCD patients is a sequela of vaso-occlusive episode [Rees, D. C., et al., 2010; Vichinsky, E. P., et al., (2000); Miller, A. C. & Gladwin, (2012)]. Histopathological findings in SCD patients [Anea, C. B., et al. (2016); Mekontso Dessap, A., et al, (2011)] and in vivo lung imaging in transgenic-humanized SCD mice [Bennewitz, M. F., et al, (2017); Vats, R., et al. (2020)] have identified that occlusion of lung arterioles (pulmonary vaso-occlusion) by neutrophil-platelet-erythrocyte aggregates promotes the development of acute chest syndrome. This epidemiology also offers a therapeutic window to prevent the development of lung injury in SCD, provided new therapies are identified to treat pulmonary vaso-occlusion. Previously, it was shown that P-selectin blockade [Bennewitz, M. F., et al. (2017)] led to partial (˜50%) protection from pulmonary vaso-occlusion in SCD mice. These findings were supported by a major clinical trial, which also reported ˜50% reduction in frequency of hospitalization among SCD patients receiving intravenous P-selectin Ab therapy [Ataga, K. I., et al. (2017)]. Altogether, these findings show that the pulmonary vaso-occlusion in SCD is probably enabled by a P-selectin-independent mechanism as well, which had not been identified until now.

### GSDMD-Inhibition Prevents P-Selectin-Independent Lung Vasoocclusion in Mice with SCD In Vivo

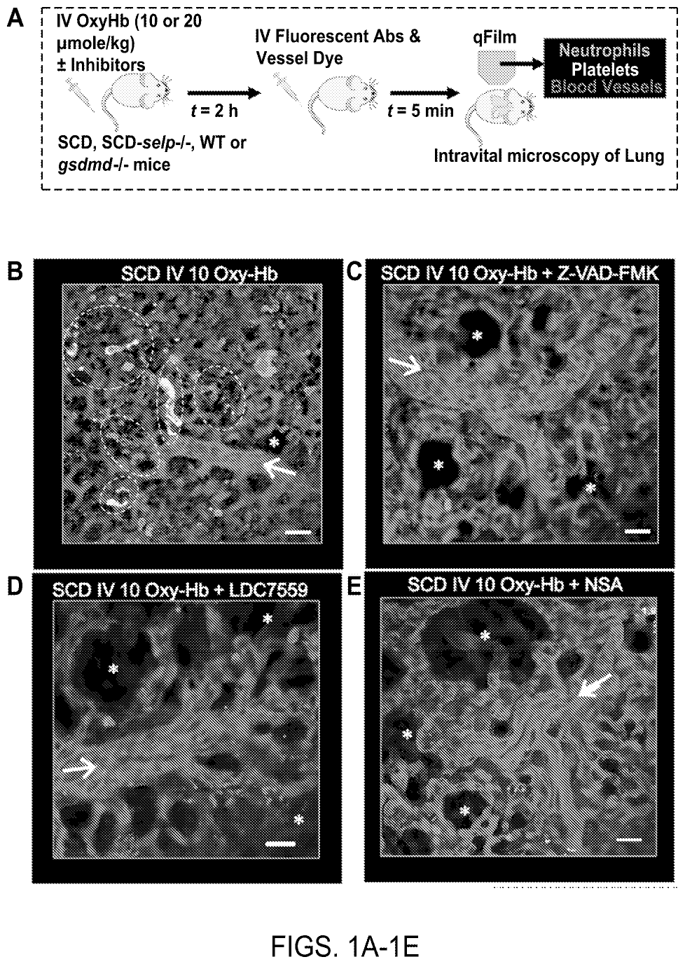

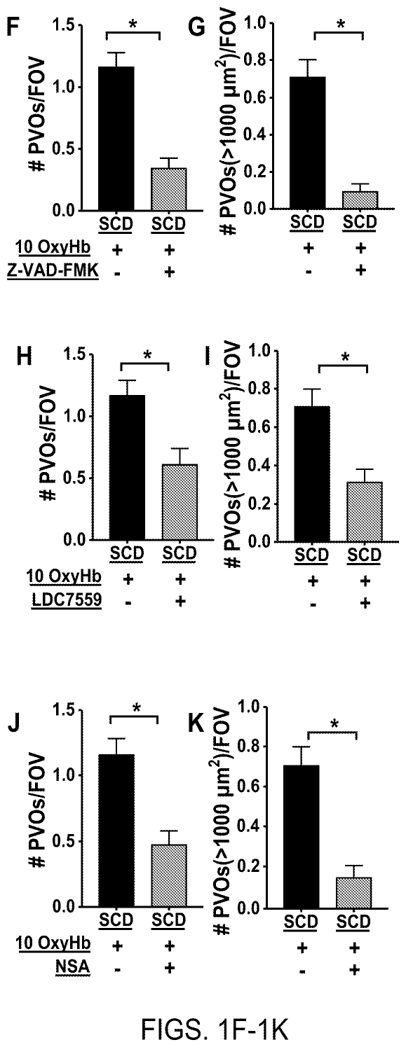

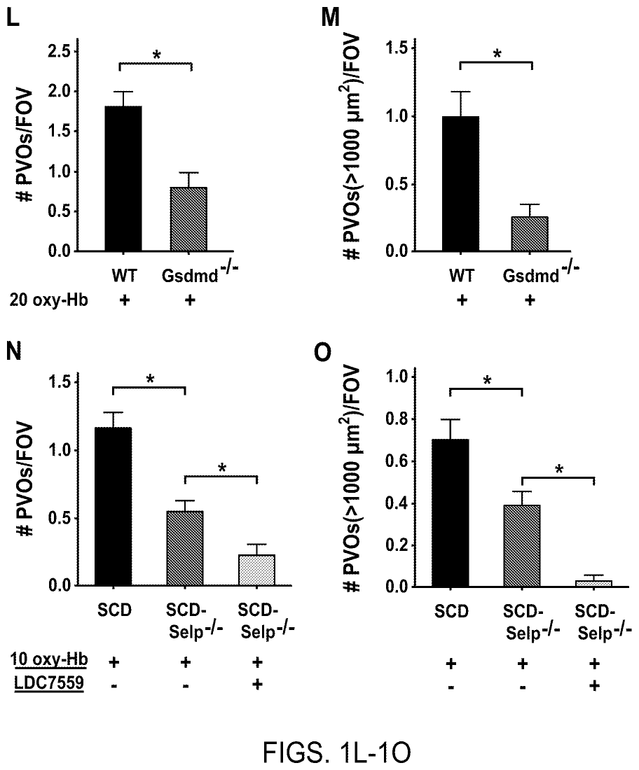

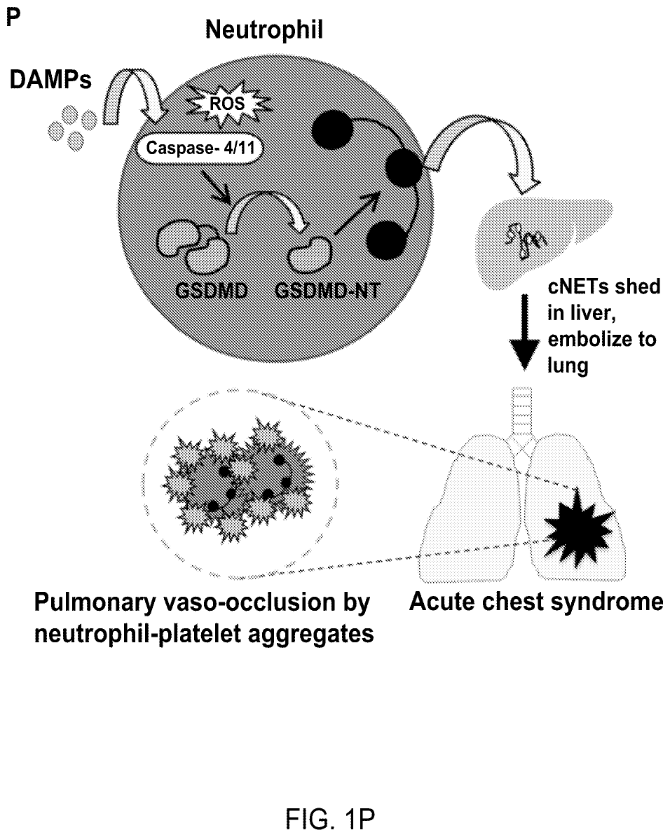

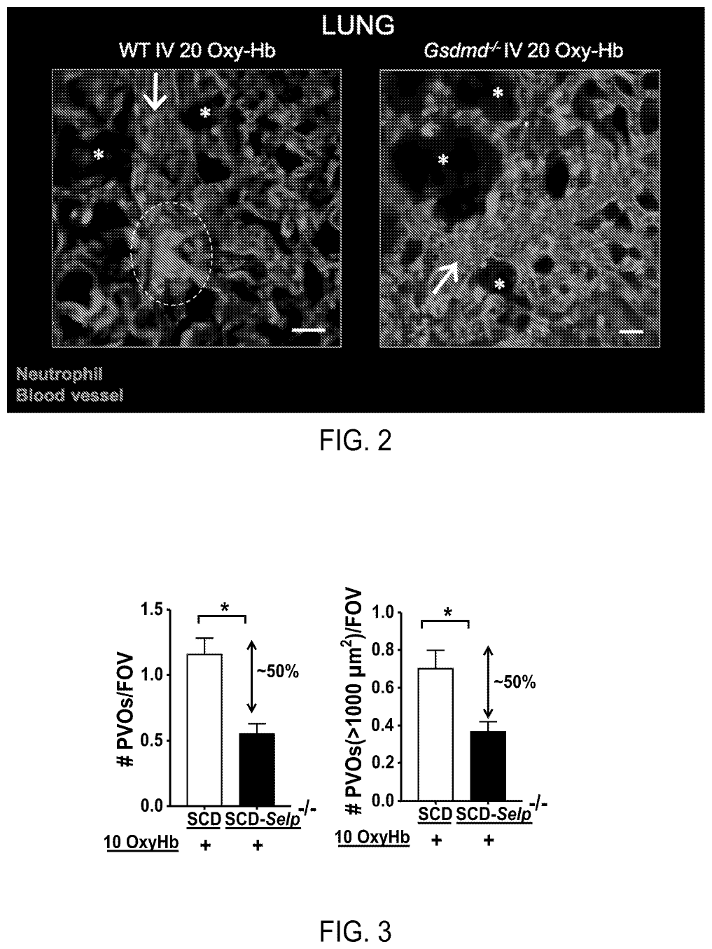

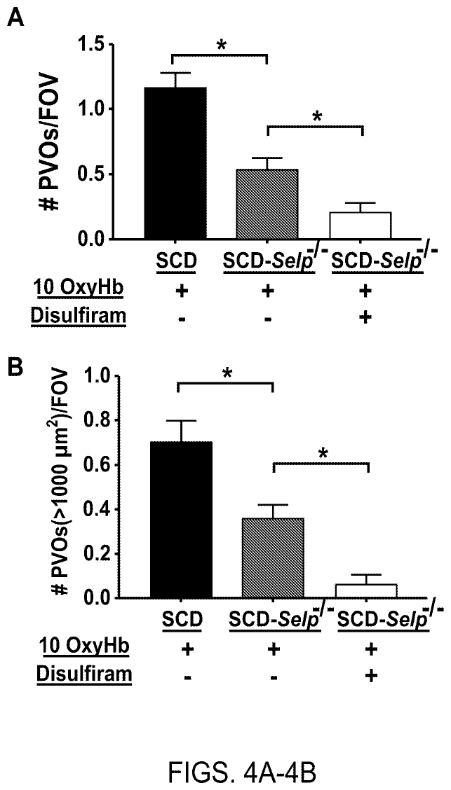

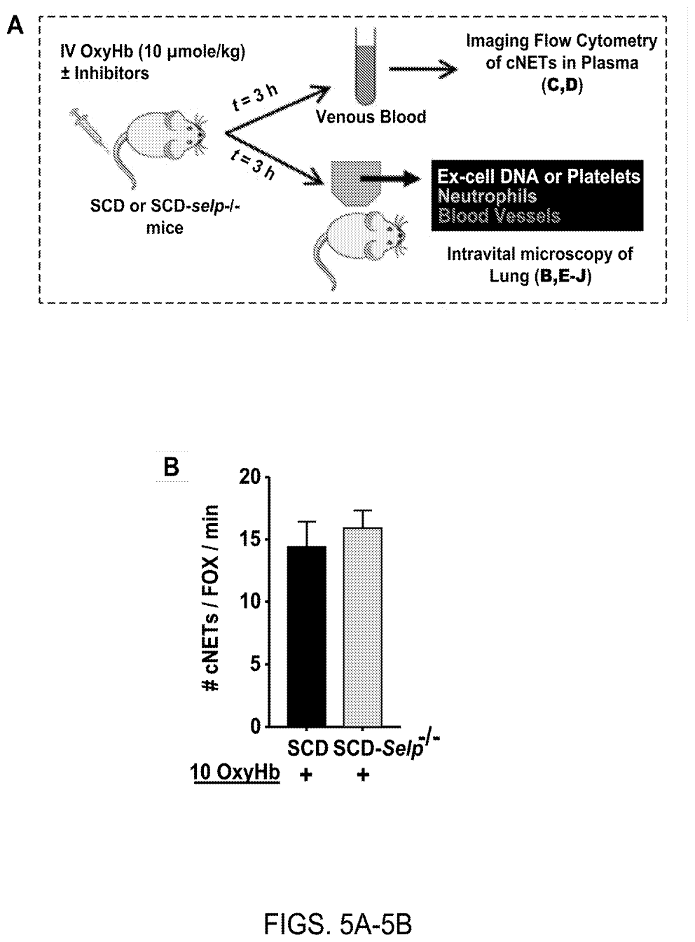

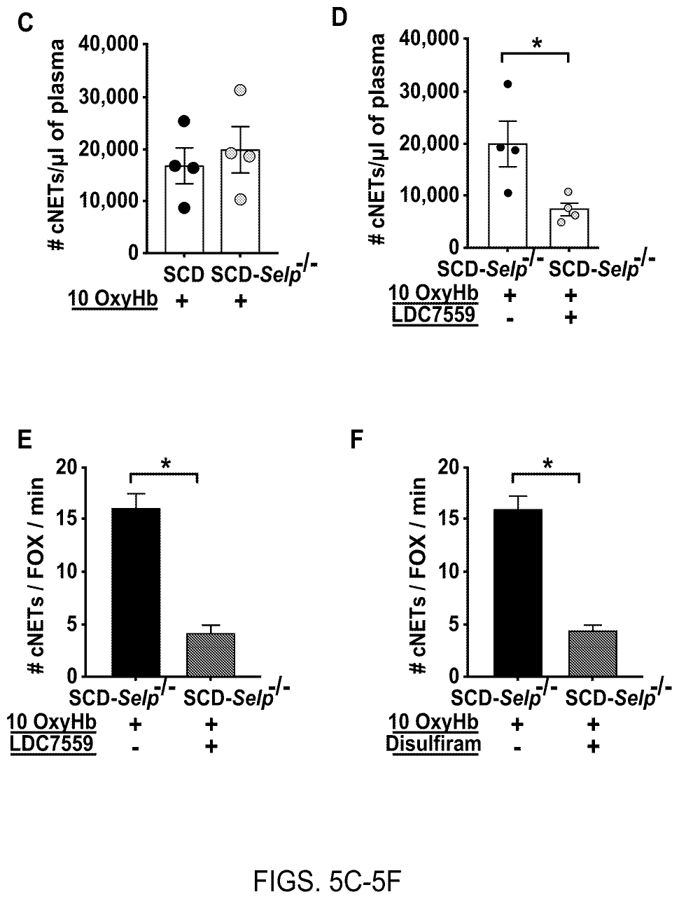

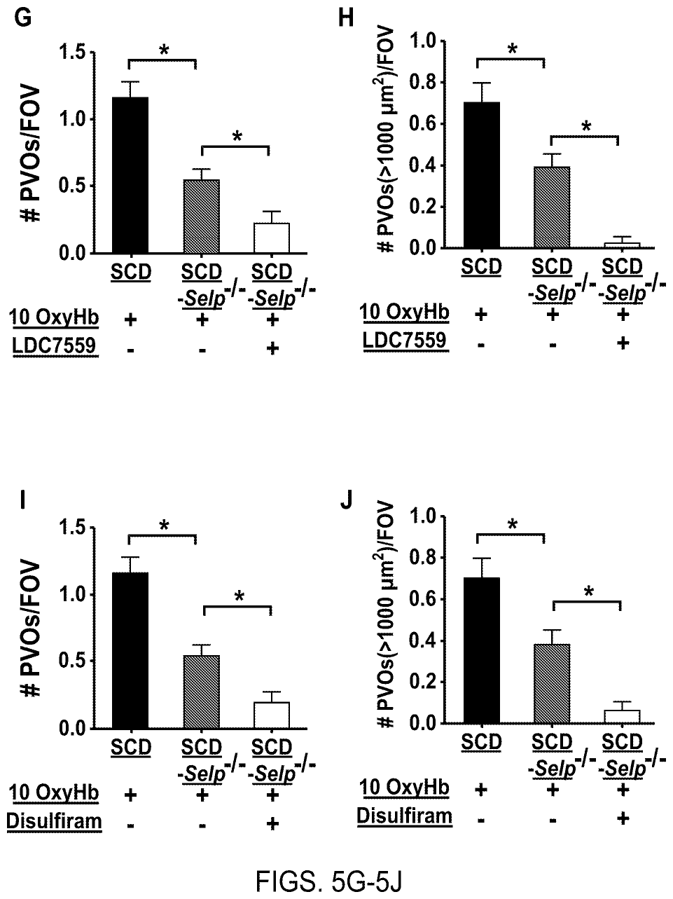

Intravital lung microscopy (experimental scheme shown in FIG. 1A) was conducted in live mice to assess whether preventing liver-to-lung embolization of cNETs by inhibiting or deleting GSDMD-signaling leads to amelioration of pulmonary vaso-occlusion. Although large neutrophil-platelet aggregates (marked with dotted ellipses) were seen occluding the arteriolar bottle-necks, leading to impaired blood flow in the lung of SCD mice IV administered 10 μmole/kg oxy-Hb (FIG. 1B), such aggregates were rare in the lung of SCD mice IV administered 10 μmole/kg oxy-Hb with 0.004 μmol/kg pan-caspase inhibitor Z-VAD-FMK (FIG. 1C) or 10 mg/kg GSDMD-NT inhibitor LDC7559 (FIG. 1D) or 20 mg/kg GSDMD-NT inhibitor Necrosulfonamide (FIG. 1E). Erythrocytes (dark cells) were observed flowing unobstructed through the pulmonary arteriole and into the pulmonary capillaries, indicating the lack of pulmonary vaso-occlusion in SCD mice IV administered oxy-Hb with Z-VAD-FMK or LDC7559 or Necrosulfonamide. The number of pulmonary vaso-occlusions per FOV and the number of large pulmonary vaso-occlusions (area>1000 μm²) per FOV were significantly reduced in SCD mice IV administered oxy-Hb with Z-VAD-FMK (FIGS. 1F, 1G) or LDC7559 (FIGS. 1H, 1I) or Necrosulfonamide (FIGS. 1J, 1K) than oxy-Hb alone. The twofold higher dose of 20 μmole/kg IV oxy-Hb also led to the development of lung vaso-occlusion in WT but not Gsdmd^{-/-} mice (FIG. 2). Both the number of pulmonary vaso-occlusions per FOV (FIG. 1L) and large pulmonary vaso-occlusions (area>1000 μm²) per FOV (FIG. 1M) were significantly less in Gsdmd^{-/-} than wild-type mice administered 20 μmole/kg IV oxy-Hb. P-selectin deficiency led to an approximately 50% reduction in pulmonary vaso-occlusion in SCD-Selp^{-/-} mice administered 10 μmole/kg IV oxy-Hb (FIG. 3), however, IV administration of oxy-Hb with GSDMD-NT inhibitor (LDC7559) led to further threefold (significant) reduction in pulmonary vaso-occlusions per FOV (FIGS. 1N and 4A, and FIG. 5) and absence of large pulmonary vaso-occlusions (area>1000 μm²) per FOV (FIG. 10) in SCD-Selp^{-/-} mice.

### Example 2. Methods and Materials

### qFILM

Quantitative fluorescence intravital lung microscopy (qFILM) has been used widely for in vivo assessment of pulmonary vasoocclusion in mice with SCD [Bennewitz M F et al., (2017); Vats R, Brzoska T, Bennewitz M F, et al. (2020); Vats R, Tutuncuoglu E, Pradhan-Sundd T, Tejero J, Shaw G D, Sundd P. (2020); Bennewitz M F, Tutuncuoglu E, Gudapati S, et al. (2020)] In the current study, qFILM was used to assess pulmonary vaso-occlusion and detect NETs in the intact lung microcirculation of live mice following IV challenge with saline, oxy-hemoglobin (oxy-Hb), hemin, or lipo-polysaccharide with or without pretreatment with N-terminus active domain (GSDMD-NT) inhibitors LDC7559 [Sollberger G, Choidas A, Burn G L, et al. (2018)], NSA [Rathkey J K, Zhao J, Liu Z, et al. (2018)], Disulfiram [Hu J J, Liu X, Xia S, et al. (2020)], or the pan-caspase inhibitor Z-VAD-FMK [Sollberger G, Choidas A, Burn G L, et al. (2018)], haptoglobin [Belcher J D, Chen C, Nguyen J, et al. (2014)], or TLR4-inhibitor (TAK242) [Belcher J D, Chen C, Nguyen J, et al. (2014)]. qFILM was conducted with a Nikon multiphoton excitation fluorescence microscope and an APO LWD 25×water immersion objective with 1.1 NA. Before imaging, mice were anesthetized with an intraperitoneal injection of ketamine HCl and xylazine. A cannula was inserted into the right carotid artery, and tracheotomy was performed to facilitate mechanical ventilation with 95% O₂ and supply maintenance anesthesia (1% to 2% isoflurane). The left lung was surgically exposed, and a small portion of the lung was immobilized against a coverslip using a vacuum-enabled micromachined device as described elsewhere [Bennewitz M F, Watkins S C, Sundd P. (2014); Brzoska T, Kaminski T W, Bennewitz M F, Sundd P. (2020)]. Next, fluorescent antibodies and dyes were injected into the carotid artery catheter for visualization of the pulmonary microcirculation, extracellular DNA, and in vivo staining of neutrophil elastase (NE), citrullinated histones, neutrophils, and platelets, respectively. Pulmonary vaso-occlusions and NETs were quantified and compared between treatment groups using the strategy described in supplemental methods and described previously [Bennewitz M F et al. (2017); Vats R, Brzoska T, Bennewitz M F, et al. (2020); Chen G et al. (2014); Vats R, Tutuncuoglu E, Pradhan-Sundd T, Tejero J, Shaw G D, Sundd P (2020); Bennewitz M F et al. (2020); Kolaczkowska E et al. (2015)].

### Intravital Liver Microscopy

The liver intravital microscopy experimental setup has been described previously in detail [Vats R, Kaminski T W, Ju E M, et al. (2021); Vats R, Liu S, Zhu J, et al. (2020); Pradhan-Sundd T et al. (2018)]. The same strains of mice and treatment groups as those used in qFILM studies were used. Mice were anesthetized with an intraperitoneal injection of ketamine HCl and xylazine. The right carotid artery was cannulated, and the right lobe of the liver was gently immobilized against a coverslip using a vacuum-enabled micromachined liver-imaging window described elsewhere [Vats R, Kaminski T W, Ju E M, et al. (2021); Vats R, Liu S, Zhu J, et al. (2020); Pradhan-Sundd T et al. (2018)]. Next, fluorescent dyes and antibodies were injected into the carotid artery catheter, and intravital observations were conducted with a Nikon multiphoton excitation fluorescence microscope.

### Statistical Analysis

Means were compared using the unpaired Student t test without or with Bonferroni correction or one-way analysis of variance with Games-Howell's multiple comparison test. Percentages were compared using the fourfold table analysis with x2 statistics. P, 0.05 was considered significant. Unpaired or paired 2-tailed Student t test was used to confirm the significance in western blot analyses.

SEQUENCES
(amino acid sequence of gasdermin-D)
SEQ ID NO: 1
MGSAFERVVRRVVQELDHGGEFIPVTSLQSSTGFQPYCLVVRKPSSSWFW
KPRYKCVNLSIKDILEPDAAEPDVQRGRSFHFYDAMDGQIQGSVELAAPG
QAKIAGGAAVSDSSSTSMNVYSLSVDPNTWQTLLHERHLRQPEHKVLQQL
RSRGDNVYVVTEVLQTQKEVEVTRTHKREGSGRESLPGATCLQGEGQGHL
SQKKTVTIPSGSTLAFRVAQLVIDSDLDVLLFPDKKQRTFQPPATGHKRS
TSEGAWPQLPSGLSMMRCLHNFLTDGVPAEGAFTEDFQGLRAEVETISKE
LELLDRELCQLLLEGLEGVLRDQLALRALEEALEQGQSLGPVEPLDGPAG
AVLECLVLSSGMLVPELAIPVVYLLGALTMLSETQHKLLAEALESQTLLG
PLELVGSLLEQSAPWQERSTMSLPPGLLGNSWGEGAPAWVLLDECGLELG
EDTPHVCWEPQAQGRMCALYASLALLSGLSQEPH
(amino acid sequence of P-selectin)
SEQ ID NO: 2
MANCQIAILYQRFQRVVFGISQLLCFSALISELTNQKEVAAWTYHYSTKA
YSWNISRKYCQNRYTDLVAIQNKNEIDYLNKVLPYYSSYYWIGIRKNNKT
WTWVGTKKALTNEAENWADNEPNNKRNNEDCVEIYIKSPSAPGKWNDEHC
LKKKHALCYTASCQDMSCSKQGECLETIGNYTCSCYPGFYGPECEYVREC
GELELPQHVLMNCSHPLGNFSFNSQCSFHCTDGYQVNGPSKLECLASGIW
TNKPPQCLAAQCPPLKIPERGNMTCLHSAKAFQHQSSCSFSCEEGFALVG
PEVVQCTASGVWTAPAPVCKAVQCQHLEAPSEGTMDCVHPLTAFAYGSSC
KFECQPGYRVRGLDMLRCIDSGHWSAPLPTCEAISCEPLESPVHGSMDCS
PSLRAFQYDTNCSFRCAEGFMLRGADIVRCDNLGQWTAPAPVCQALQCQD
LPVPNEARVNCSHPFGAFRYQSVCSFTCNEGLLLVGASVLQCLATGNWNS
VPPECQAIPCTPLLSPQNGTMTCVQPLGSSSYKSTCQFICDEGYSLSGPE
RLDCTRSGRWTDSPPMCEAIKCPELFAPEQGSLDCSDTRGEFNVGSTCHF
SCDNGFKLEGPNNVECTTSGRWSATPPTCKGIASLPTPGLQCPALTTPGQ
GTMYCRHHPGTFGFNTTCYFGCNAGFTLIGDSTLSCRPSGQWTAVTPACR
AVKCSELHVNKPIAMNCSNLWGNFSYGSICSFHCLEGQLLNGSAQTACQE
NGHWSTTVPTCQAGPLTIQEALTYFGGAVASTIGLIMGGTLLALLRKRFR
QKDDGKCPLNPHSHLGTYGVFTNAAFDPSP

1. A method of treating pulmonary vaso-occlusion in a subject in need thereof, comprising administering a therapeutically effective amount of a gasdermin-D inhibitor to the subject.

2. The method of claim 1, wherein the subject has sickle cell disease.

3. The method of claim 1, wherein the gasdermin-D inhibitor is selected from the group consisting of LDC7559, necrosulfonamide, and disulfiram.

4. The method of claim 1, wherein the gasdermin-D inhibitor is a gasdermin-D gene editing tool.

5. The method of claim 4, wherein the gasdermin-D gene editing tool is a small interfering RNA (siRNA), a short hairpin RNA (shRNA), CRISPR-Cas9, or CRISPR-Cas13.

6. The method of claim 1, further comprising administering to the subject a therapeutically effective amount of a P-selectin inhibitor.

7. The method of claim 6, wherein the P-selectin inhibitor is an antibody that specifically binds to P-selectin.

8. The method of claim 7, wherein the P-selectin inhibitor is crizanlizumab.

9. The method of claim 6, wherein the P-selectin inhibitor is administered concurrently or sequentially with the gasdermin-D inhibitor.

10. The method of claim 9, wherein the subject is administered the P-selectin inhibitor prior to the administration of the gasdermin-D inhibitor.

11. The method of claim 10, wherein the subject is resistant to the P-selectin inhibitor.

12. A method of treating pulmonary vaso-occlusion in a subject in need thereof, comprising administering a therapeutically effective amount of a gasdermin-D inhibitor to the subject, wherein the subject is resistant to a P-selectin inhibitor.

13. The method of claim 12, wherein the subject has sickle cell disease.

14. The method of claim 12, wherein the gasdermin-D inhibitor is selected from the group consisting of LDC7559, necrosulfonamide, and disulfiram.

15. The method of claim 12, wherein the gasdermin-D inhibitor is a gasdermin-D gene editing tool.

16. The method of claim 15, wherein the gasdermin-D gene editing tool is a small interfering RNA (siRNA), a short hairpin RNA (shRNA), CRISPR-Cas9, or CRISPR-Cas13.

17. The method of claim 12, wherein the P-selectin inhibitor is an antibody that specifically binds to P-selectin.

18. The method of claim 17, wherein the P-selectin inhibitor is crizanlizumab.
